SEQUENCE LISTING (4 sequences)

SEQ ID NO: 1 - DNA, 26 bp, Maraba virus
gagatcgatctgtttacgcgtcacta

SEQ ID NO: 2 - DNA, 27 bp, Maraba virus
aatctgttgtgcaggatttgagttatt

SEQ ID NO: 3 - DNA, 27 bp, Vesicular stomatitis virus
gagtcgattgggaaataaataactcaa

SEQ ID NO: 4 - DNA, 20 bp, Vesicular stomatitis virus
gctgaacttgtggccgttta

(19) United States
(12) Patent Application Publication
(10) Pub. No.: US 2014/0271564 A1
(43) Pub. Date: Sep. 18, 2014

(54) VESICULAR STOMATITIS VIRUSES CONTAINING A MARABA VIRUS GLYCOPROTEIN POLYPEPTIDE

(71) Applicant: Mayo Foundation for Medical Education and Research, Rochester, MN (US)

(72) Inventors: Stephen James Russell, Rochester, MN (US); Mulu Tesfay, Rochester, MN (US)

(21) Appl. No.: 14/204,768

(22) Filed: Mar. 11, 2014

Related U.S. Application Data
(60) Provisional application No. 61/780,353, filed on Mar. 13, 2013.

(51) Int. Cl. A61K 35/76

(52) U.S. Cl. CPC A61K 35/768
USPC 424/93.6; 435/235.1; 536/23.72

(57) ABSTRACT

This document provides methods and materials related to vesicular stomatitis viruses containing a G polypeptide of a maraba virus. For example, vesicular stomatitis viruses containing a G polypeptide of a maraba virus (e.g., pseudotyped viruses), nucleic acid molecules encoding vesicular stomatitis viruses containing a G polypeptide of a maraba virus, methods for making vesicular stomatitis viruses containing a G polypeptide of a maraba virus, and methods for using vesicular stomatitis viruses containing a G polypeptide of a maraba virus to treat cancer or infectious diseases are provided.

### CROSS-REFERENCE TO RELATED APPLICATIONS

This application claims the benefit of U.S. Application Ser. No. 61/780,353, filed Mar. 13, 2013. The disclosure of the prior application is considered part of (and is incorporated by reference in) the disclosure of this application.

### BACKGROUND

1. Technical Field

This document relates to methods and materials involved in making and using vesicular stomatitis viruses containing a glycoprotein (G) polypeptide of a maraba virus. For example, this document relates to vesicular stomatitis viruses containing a G polypeptide of a maraba virus, nucleic acid molecules encoding vesicular stomatitis viruses containing a G polypeptide of a maraba virus, methods for making vesicular stomatitis viruses containing a G polypeptide of a maraba virus, and methods for using such vesicular stomatitis viruses containing a G polypeptide of a maraba virus to treat cancer (e.g., multiple myeloma) or infectious diseases.

2. Background Information

Vesicular stomatitis virus (VSV) is a member of the Rhabdoviridae family. The VSV genome is a single molecule of negative-sense RNA that encodes five major polypeptides: a nucleocapsid (N) polypeptide, a phosphoprotein (P) polypeptide, a matrix (M) polypeptide, a glycoprotein (G) polypeptide, and a viral polymerase (L) polypeptide.

### SUMMARY

This document provides methods and materials related to vesicular stomatitis viruses containing a G polypeptide of a maraba virus. For example, this document provides vesicular stomatitis viruses containing a G polypeptide of a maraba virus (e.g., pseudotyped viruses), nucleic acid molecules encoding vesicular stomatitis viruses containing a G polypeptide of a maraba virus, methods for making vesicular stomatitis viruses containing a G polypeptide of a maraba virus, and methods for using vesicular stomatitis viruses containing a G polypeptide of a maraba virus to treat cancer such as multiple myeloma or infectious diseases such as HIV, Ebola virus, or human respiratory syncytial virus (RSV) infections.

As described herein, pseudotyped vesicular stomatitis viruses can be designed to have a G polypeptide of a maraba virus in place of a VSV G polypeptide. In some cases, such pseudotyped vesicular stomatitis viruses can include a nucleic acid molecule that encodes a VSV N polypeptide, a VSV P polypeptide, a VSV M polypeptide, and a VSV L polypeptide. Such a nucleic acid molecule can lack (a) a functional VSV G polypeptide and/or the nucleic acid sequence that encodes a full-length VSV G polypeptide and (b) a functional maraba G polypeptide and/or the nucleic acid sequence that encodes a full-length maraba G polypeptide. In some cases, a vesicular stomatitis virus containing a G polypeptide of a maraba virus (e.g., a pseudotyped vesicular stomatitis virus containing a G polypeptide of a maraba virus) can be replication incompetent.

In some cases, vesicular stomatitis viruses can be designed to have a nucleic acid molecule that encodes a VSV N polypeptide, a VSV P polypeptide, a VSV M polypeptide, a maraba G polypeptide, and a VSV L polypeptide. Such a nucleic acid molecule can lack a functional VSV G polypeptide and/or lack the nucleic acid sequence that encodes a full-length VSV G polypeptide. For example, a vesicular stomatitis virus provided herein can be designed to have a nucleic acid molecule that encodes a VSV N polypeptide, a VSV P polypeptide, a VSV M polypeptide, a maraba G polypeptide, and a VSV L polypeptide and lacks the ability to encode a functional VSV G polypeptide. In some cases, a vesicular stomatitis virus provided herein can be designed to have a nucleic acid molecule that encodes a VSV N polypeptide, a VSV P polypeptide, a VSV M polypeptide, a maraba G polypeptide, and a VSV L polypeptide with the nucleic acid sequence encoding the maraba G polypeptide being located in the position where the nucleic acid sequence encoding a full-length VSV G polypeptide is normally located in a wild-type vesicular stomatitis virus. In some cases, a vesicular stomatitis virus provided herein can be designed to have a nucleic acid molecule where the nucleic acid sequence encoding a VSV G polypeptide is replaced with nucleic acid that encodes a maraba G polypeptide. In some cases, a vesicular stomatitis virus designed to have a nucleic acid molecule that encodes a VSV N polypeptide, a VSV P polypeptide, a VSV M polypeptide, a maraba G polypeptide, and a VSV L polypeptide can be replication competent.

As described herein, vesicular stomatitis viruses containing a G polypeptide of a maraba virus (e.g., pseudotyped vesicular stomatitis viruses designed to have a G polypeptide of a maraba virus in place of a VSV G polypeptide) can be designed to retain the cell tropism and replication kinetics of wild-type vesicular stomatitis viruses. In some cases, vesicular stomatitis viruses containing a G polypeptide of a maraba virus can be designed to have increased resistance to neutralization by both non-immune and VSV-immune sera as compared to wild-type vesicular stomatitis viruses.

In some cases, a vesicular stomatitis virus provided herein can have a nucleic acid molecule that includes a sequence encoding an interferon (IFN) polypeptide (e.g., a human IFN-β polypeptide), a sodium iodide symporter (NIS) polypeptide (e.g., a human NIS polypeptide), a fluorescent polypeptide (e.g., a GFP polypeptide), any appropriate therapeutic transgene (e.g., HSV-TK or cytosine deaminase), a polypeptide that antagonizes host immunity (e.g., influenza NS1, HSVγ34.5, or SOCS1), or a tumor antigen (e.g., cancer vaccine components). The nucleic acid encoding the IFN polypeptide can be positioned between the nucleic acid encoding the VSV M polypeptide and the nucleic acid encoding the VSV L polypeptide. Such a position can allow the viruses to express an amount of the IFN polypeptide that is effective to activate anti-viral innate immune responses in non-cancerous tissues, and thus alleviate potential viral toxicity, without impeding efficient viral replication in cancer cells for those vesicular stomatitis viruses provided herein that are replication competent. The nucleic acid encoding the NIS polypeptide can be positioned between the nucleic acid encoding the VSV M polypeptide and the VSV L polypeptide. Such a position of can allow the viruses to express an amount of the NIS polypeptide that (a) is effective to allow selective accumulation of iodide (and/or other ligands transported by NIS polypeptides such as perchlorate, pertechnetate, and tetrafluoroborate) in infected cells, thereby allowing both imaging of viral distribution using radioisotopes and radiotherapy targeted to infected cancer cells, and (b) is not so high as to be toxic to infected cells. In some cases, for replication competent vesicular stomatitis virus provided herein, positioning the nucleic acid encoding an IFN polypeptide between the nucleic acid encoding the VSV M polypeptide and the nucleic acid encoding the VSV L polypeptide and positioning the nucleic acid encoding a NIS polypeptide between the nucleic acid encoding the VSV M polypeptide and the VSV L polypeptide within the genome of a vesicular stomatitis virus can result in vesicular stomatitis viruses that are viable, that have the ability to replicate and spread, that express appropriate levels of functional IFN polypeptides, and that expression appropriate levels of functional NIS polypeptides to take up radio-iodine for both imaging and radio-virotherapy.

In general, one aspect of this document features a replication-incompetent vesicular stomatitis virus comprising a maraba G polypeptide and an RNA molecule. The RNA molecule comprises, or consists essentially of, a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The RNA molecule can comprise a nucleic acid sequence that is a template for a positive sense transcript encoding a NIS polypeptide.

In another aspect, this document features a replication-competent vesicular stomatitis virus comprising an RNA molecule. The RNA molecule comprises, or consists essentially of, a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The RNA molecule can comprise a nucleic acid sequence that is a template for a positive sense transcript encoding a NIS polypeptide.

In another aspect, this document features a composition comprising a replication-incompetent vesicular stomatitis virus comprising a maraba G polypeptide and an RNA molecule. The RNA molecule comprises, or consists essentially of, a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The RNA molecule can comprise a nucleic acid sequence that is a template for a positive sense transcript encoding a NIS polypeptide.

In another aspect, this document features a composition comprising a replication-competent vesicular stomatitis virus comprising an RNA molecule. The RNA molecule comprises, or consists essentially of, a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The RNA molecule can comprise a nucleic acid sequence that is a template for a positive sense transcript encoding a NIS polypeptide.

In another aspect, this document features a nucleic acid molecule comprising, or consisting essentially of, a nucleic acid strand comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the nucleic acid strand lacks a nucleic acid sequence that is a template for a positive sense transcript encoding a functional VSV G polypeptide. The nucleic acid strand can comprise a nucleic acid sequence that is a template for a positive sense transcript encoding a NIS polypeptide. The NIS polypeptide can be a human NIS polypeptide.

In another aspect, this document features a method for treating cancer. The method comprises, or consists essentially of, administering a composition comprising replication-competent vesicular stomatitis viruses to a mammal comprising cancer cells, wherein the vesicular stomatitis viruses comprise an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide, wherein administration of the composition to the mammal is under conditions wherein the vesicular stomatitis viruses infect the cancer cells to form infected cancer cells, and wherein the number of cancer cells within the mammal is reduced following the administration. The mammal can be a human. The RNA molecule can comprise a nucleic acid sequence that is a template for a positive sense transcript encoding a NIS polypeptide. The NIS polypeptide can be a human NIS polypeptide. The cancer can be multiple myeloma.

In another aspect, this document features a method for inducing tumor regression in a mammal. The method comprises, or consists essentially of, administering a composition comprising replication-competent vesicular stomatitis viruses to a mammal comprising a tumor, wherein the vesicular stomatitis viruses comprise an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide, wherein administration of the composition to the mammal is under conditions wherein the vesicular stomatitis viruses infect tumor cells of the tumor to form infected tumor cells. The mammal can be a human. The RNA molecule can comprise a nucleic acid sequence that is a template for a positive sense transcript encoding a NIS polypeptide. The NIS polypeptide can be a human NIS polypeptide. The tumor can be multiple myeloma.

In another aspect, this document features a method for rescuing replication-competent vesicular stomatitis viruses from cells. The vesicular stomatitis viruses comprises, or consists essentially of, an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The method comprises, or consists essentially of, (a) inserting nucleic acid encoding the RNA molecule into the cells under conditions wherein replication-competent vesicular stomatitis viruses are produced, and (b) harvesting the replication-competent vesicular stomatitis viruses.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of the replacement of VSV G nucleic acid with nucleic acid encoding a G polypeptide of a maraba virus.

FIG. 2 contains photographs of different cell types exposed to VSV or VSV/MarabaG viruses. Pictures were taken 24 hours post-infection. These results demonstrate that vesicular stomatitis viruses containing with a G polypeptide of a maraba virus (VSV/MaravG) have similar virus infectivity and tropism as wild-type vesicular stomatitis viruses (pVSV).

FIG. 3 is a graph plotting the virus replication kinetics of vesicular stomatitis viruses containing a G polypeptide of a maraba virus (VSV/MaravG) and wild-type vesicular stomatitis viruses (pVSV).

FIG. 4 contains photographs of cells exposed to wild type VSV or VSV/MarabaG or wild type Maraba virus in the presence or absence of human non-immune serum (NHS) or VSV-immune serum. These results demonstrate that vesicular stomatitis viruses containing a G polypeptide of a maraba virus (VSV/MaravG) escape serum neutralization.

FIG. 5 is a graph plotting the viral titers (fifty percent tissue culture infectious dose (TCID₅₀/mL) values) of wild-type VSV or VSV/MarabaG virus in the presence or absence of non-immune human serum or VSV-immune serum. A medium treated control is included.

FIG. 6 contains graphs plotting the viral titers (TCID₅₀/mL values) of wild-type VSV (VSV), VSV/MarabaG (VSV/MarabaG), or Maraba (Maraba) viruses in the presence of medium (Medium), non-immune human serum (NHS), VSV-immune mouse serum (VSV-immune serum as indicated), non-immune dog serum (NDS-1 and NDS-2), non-immune simian monkey serum (NMS-1 and NMS-2), or VSV-immune human serum (VSV-immune serum as indicated). VSV/Maraba G resisted neutralization, both by normal human serum (NHS) and VSV-immune sera from different origins.

FIG. 7 contains photographs of cells demonstrating that normal human serum inhibits VSV, but not VSV/MarG, infection in multiple cell lines.

FIG. 8 contains photographs demonstrating that VSV, Maraba, and VSV/MarG viruses possess similar receptor requirements. MV represents measles viruses.

FIG. 9 is a graph plotting the percent survival of mice having multiple myeloma and treated with PBS, wild-type VSV viruses, or VSV/MarG viruses. These in vivo results demonstrate that multiple myeloma mice treated with VSV/MarG viruses exhibit superior survival as compared to multiple myeloma mice treated with wild-type VSV viruses in the presence of anti-VSV antibodies.

### DETAILED DESCRIPTION

This document provides methods and materials related to vesicular stomatitis viruses containing a G polypeptide of a maraba virus. For example, this document provides vesicular stomatitis viruses containing a G polypeptide of a maraba virus (e.g., pseudotyped viruses), nucleic acid molecules encoding vesicular stomatitis viruses containing a G polypeptide of a maraba virus, methods for making vesicular stomatitis viruses containing a G polypeptide of a maraba virus, and methods for using vesicular stomatitis viruses containing a G polypeptide of a maraba virus to treat cancer such as or infectious diseases.

As described herein, pseudotyped vesicular stomatitis viruses can be designed to have a G polypeptide of a maraba virus in place of a VSV G polypeptide. In some cases, such pseudotyped vesicular stomatitis viruses can include a nucleic acid molecule that encodes a VSV N polypeptide, a VSV P polypeptide, a VSV M polypeptide, and a VSV L polypeptide. Such a nucleic acid molecule can lack (a) a functional VSV G polypeptide and/or the nucleic acid sequence that encodes a full-length VSV G polypeptide and (b) a functional maraba G polypeptide and/or the nucleic acid sequence that encodes a full-length maraba G polypeptide. In some cases, a vesicular stomatitis virus containing a G polypeptide of a maraba virus (e.g., a pseudotyped vesicular stomatitis virus containing a G polypeptide of a maraba virus) can be replication incompetent.

In some cases, a vesicular stomatitis virus provided herein can be designed to have a nucleic acid molecule that encodes a VSV N polypeptide, a VSV P polypeptide, a VSV M polypeptide, a maraba G polypeptide, and a VSV L polypeptide, and that does not encode a functional VSV G polypeptide. It will be appreciated that the sequences described herein with respect to a vesicular stomatitis virus are incorporated into a plasmid coding for the positive sense cDNA of the viral genome allowing generation of the negative sense genome of vesicular stomatitis viruses. Thus, it will be appreciated that a nucleic acid sequence that encodes a VSV polypeptide, for example, can refer to an RNA sequence that is the template for the positive sense transcript that encodes (e.g., via direct translation) that polypeptide.

The nucleic acid encoding a maraba G polypeptide can be positioned at any location within the VSV genome. In some cases, the nucleic acid encoding a maraba G polypeptide can be positioned downstream of the nucleic acid encoding the VSV M polypeptide. For example, nucleic acid encoding a maraba G polypeptide can be positioned between nucleic acid encoding a VSV M polypeptide and nucleic acid encoding a VSV L polypeptide.

Any appropriate nucleic acid encoding a maraba G polypeptide can be inserted into the genome of a vesicular stomatitis virus. For example, nucleic acid encoding a wild-type maraba G polypeptide can be inserted into the genome of a vesicular stomatitis virus. Examples of nucleic acid encoding a maraba G polypeptide that can be inserted into the genome of a vesicular stomatitis virus include, without limitation, nucleic acid encoding a maraba G polypeptide set forth in GenBank® Accession No. FW339541.1 (GI No. 298563846).

In some cases, the nucleic acid molecule of vesicular stomatitis virus provided herein can encode an IFN polypeptide, a fluorescent polypeptide (e.g., a GFP polypeptide), a NIS polypeptide, a therapeutic polypeptide, an innate immunity antagonizing polypeptide, a tumor antigen, or a combination thereof. Nucleic acid encoding an IFN polypeptide can be positioned downstream of nucleic acid encoding a VSV M polypeptide. For example, nucleic acid encoding an IFN polypeptide can be positioned between nucleic acid encoding a VSV M polypeptide and nucleic acid encoding a maraba G polypeptide. Such a position can allow the viruses to express an amount of IFN polypeptide that is effective to activate anti-viral innate immune responses in non-cancerous tissues, and thus alleviate potential viral toxicity, without impeding efficient viral replication in cancer cells.

Any appropriate nucleic acid encoding an IFN polypeptide can be inserted into the genome of a vesicular stomatitis virus. For example, nucleic acid encoding an IFN beta polypeptide can be inserted into the genome of a vesicular stomatitis virus. Examples of nucleic acid encoding IFN beta polypeptides that can be inserted into the genome of a vesicular stomatitis virus include, without limitation, nucleic acid encoding a human IFN beta polypeptide of the nucleic acid sequence set forth in GenBank® Accession No. NM_{—}002176.2 (GI No. 50593016), nucleic acid encoding a mouse IFN beta polypeptide of the nucleic acid sequence set forth in GenBank® Accession Nos. NM_{—}010510.1 (GI No. 6754303), BC119395.1 (GI No. 111601321), or BC119397.1 (GI No. 111601034), and nucleic acid encoding a rat IFN beta polypeptide of the nucleic acid sequence set forth in GenBank® Accession No. NM_{—}019127.1 (GI No. 9506800).

Nucleic acid encoding a NIS polypeptide can be positioned downstream of nucleic acid encoding a maraba G polypeptide. For example, nucleic acid encoding a NIS polypeptide can be positioned between nucleic acid encoding a maraba G polypeptide and nucleic acid encoding a VSV L polypeptide. Such a position of can allow the viruses to express an amount of NIS polypeptide that (a) is effective to allow selective accumulation of iodide (and/or other ligands transported by NIS polypeptides such as perchlorate, pertechnetate, and tetrafluoroborate) in infected cells, thereby allowing both imaging of viral distribution using radioisotopes and radiotherapy targeted to infected cancer cells, and (b) is not so high as to be toxic to infected cells.

Any appropriate nucleic acid encoding a NIS polypeptide can be inserted into the genome of a vesicular stomatitis virus. For example, nucleic acid encoding a human NIS polypeptide can be inserted into the genome of a vesicular stomatitis virus. Examples of nucleic acid encoding NIS polypeptides that can be inserted into the genome of a vesicular stomatitis virus include, without limitation, nucleic acid encoding a human NIS polypeptide of the nucleic acid sequence set forth in GenBank® Accession Nos. NM_{—}000453.2 (GI No. 164663746), BC105049.1 (GI No. 85397913), or BC105047.1 (GI No. 85397519), nucleic acid encoding a mouse NIS polypeptide of the nucleic acid sequence set forth in GenBank® Accession Nos. NM_{—}053248.2 (GI No. 162138896), AF380353.1 (GI No. 14290144), or AF235001.1 (GI No. 12642413), nucleic acid encoding a chimpanzee NIS polypeptide of the nucleic acid sequence set forth in GenBank® Accession No. XM_{—}524154 (GI No. 114676080), nucleic acid encoding a dog NIS polypeptide of the nucleic acid sequence set forth in GenBank® Accession No. XM_{—}541946 (GI No. 73986161), nucleic acid encoding a cow NIS polypeptide of the nucleic acid sequence set forth in GenBank® Accession No. XM_{—}581578 (GI No. 297466916), nucleic acid encoding a pig NIS polypeptide of the nucleic acid sequence set forth in GenBank® Accession No. NM_{—}214410 (GI No. 47523871), and nucleic acid encoding a rat NIS polypeptide of the nucleic acid sequence set forth in GenBank®Accession No. NM_{—}052983 (GI No. 158138504).

The nucleic acid sequences of a vesicular stomatitis virus provided herein that encode a VSV N polypeptide, a VSV P polypeptide, a VSV M polypeptide, and a VSV L polypeptide can be from a VSV Indiana strain as set forth in GenBank® Accession Nos. NC_{—}001560 (GI No. 9627229) or can be from a VSV New Jersey strain.

In one aspect, this document provides vesicular stomatitis viruses containing a nucleic acid molecule (e.g., an RNA molecule) having (e.g., in a 3′ to 5′ direction) a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide while lacking a nucleic acid sequence that is a template for a positive sense transcript encoding a functional VSV G polypeptide. Such vesicular stomatitis viruses can infect cells (e.g., cancer cells) and can be replication-competent.

Any appropriate method can be used to insert nucleic acid (e.g., nucleic acid encoding a maraba G polypeptide, nucleic acid encoding an IFN polypeptide, and/or nucleic acid encoding a NIS polypeptide) into the genome of a vesicular stomatitis virus. For example, the methods described elsewhere (Schnell et. al., PNAS, 93:11359-11365 (1996), Obuchi et al., J. Virol., 77(16):8843-56 (2003)); Goel et al., Blood, 110(7):2342-50 (2007)); and Kelly et al., J. Viral., 84(3):1550-62 (2010)) can be used to insert nucleic acid into the genome of a vesicular stomatitis virus. Any appropriate method can be used to identify vesicular stomatitis viruses containing a nucleic acid molecule described herein. Such methods include, without limitation, PCR and nucleic acid hybridization techniques such as Northern and Southern analysis. In some cases, immunohistochemistry and biochemical techniques can be used to determine if a vesicular stomatitis virus contains a particular nucleic acid molecule by detecting the expression of a polypeptide encoded by that particular nucleic acid molecule.

In another aspect, this document provides nucleic acid molecules that encode a VSV N polypeptide, a VSV P polypeptide, a VSV M polypeptide, a maraba G polypeptide, and a VSV L polypeptide, while lacking the ability to encode a functional VSV G polypeptide. For example, a nucleic acid molecule provided herein can be a single nucleic acid molecule that includes a nucleic acid sequence that encodes a VSV N polypeptide, a nucleic acid sequence that encodes a VSV P polypeptide, a nucleic acid sequence that encodes a VSV M polypeptide, a nucleic acid sequence that encodes a maraba G polypeptide, and a nucleic acid sequence that encodes a VSV L polypeptide, while lacking a nucleic acid sequence that encodes a functional VSV G polypeptide.

In another aspect, this document provides nucleic acid molecules that encode a VSV N polypeptide, a VSV P polypeptide, a VSV M polypeptide, an IFN polypeptide, a maraba G polypeptide, a NIS polypeptide, and a VSV L polypeptide, while lacking the ability to encode a functional VSV G polypeptide. For example, a nucleic acid molecule provided herein can be a single nucleic acid molecule that includes a nucleic acid sequence that encodes a VSV N polypeptide, a nucleic acid sequence that encodes a VSV P polypeptide, a nucleic acid sequence that encodes a VSV M polypeptide, a nucleic acid sequence that encodes an IFN polypeptide, a nucleic acid sequence that encodes a maraba G polypeptide, a nucleic acid sequence that encodes a NIS polypeptide, and a nucleic acid sequence that encodes a VSV L polypeptide, while lacking the ability to encode a functional VSV G polypeptide.

The term “nucleic acid” as used herein encompasses both RNA (e.g., viral RNA) and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

This document also provides method for treating cancer (e.g., to reduce tumor size, inhibit tumor growth, or reduce the number of viable tumor cells), methods for inducing host immunity against cancer, and methods for treating an infectious disease such as HIV, Ebola virus, or human RSV infections. For example, a vesicular stomatitis virus provided herein can be administered to a mammal having cancer to reduce tumor size, to inhibit cancer cell or tumor growth, to reduce the number of viable cancer cells within the mammal, and/or to induce host immunogeneic responses against a tumor. A vesicular stomatitis virus provided herein can be propagated in host cells in order to increase the available number of copies of that virus, typically by at least 2-fold (e.g., by 5- to 10-fold, by 50- to 100-fold, by 500- to 1,000-fold, or even by as much as 5,000- to 10,000-fold). In some cases, a vesicular stomatitis virus provided herein can be expanded until a desired concentration is obtained in standard cell culture media (e.g., DMEM or RPMI-1640 supplemented with 5-10% fetal bovine serum at 37° C. in 5% CO₂). A viral titer typically is assayed by inoculating cells (e.g., BHK cells, Vero cells, or 293T cells) in culture.

Vesicular stomatitis viruses provided herein can be administered to a cancer patient by, for example, direct injection into a group of cancer cells (e.g., a tumor) or intravenous delivery to cancer cells. A vesicular stomatitis virus provided herein can be used to treat different types of cancer including, without limitation, myeloma (e.g., multiple myeloma), melanoma, glioma, lymphoma, mesothelioma, and cancers of the lung, brain, stomach, colon, rectum, kidney, prostate, ovary, breast, pancreas, liver, and head and neck.

Vesicular stomatitis viruses provided herein can be administered to a patient in a biologically compatible solution or a pharmaceutically acceptable delivery vehicle, by administration either directly into a group of cancer cells (e.g., intratumorally) or systemically (e.g., intravenously). Suitable pharmaceutical formulations depend in part upon the use and the route of entry, e.g., transdermal or by injection. Such forms should not prevent the composition or formulation from reaching a target cell (i.e., a cell to which the virus is desired to be delivered to) or from exerting its effect. For example, pharmacological compositions injected into the blood stream should be soluble.

While dosages administered will vary from patient to patient (e.g., depending upon the size of a tumor), an effective dose can be determined by setting as a lower limit the concentration of virus proven to be safe and escalating to higher doses of up to 10¹² pfu, while monitoring for a reduction in cancer cell growth along with the presence of any deleterious side effects. A therapeutically effective dose typically provides at least a 10% reduction in the number of cancer cells or in tumor size. Escalating dose studies can be used to obtain a desired effect for a given viral treatment (see, e.g., Nies and Spielberg, “Principles of Therapeutics,” In Goodman & Gilman's The Pharmacological Basis of Therapeutics, eds. Hardman, et al., McGraw-Hill, NY, 1996, pp 43-62).

Vesicular stomatitis viruses provided herein can be delivered in a dose ranging from, for example, about 10³ pfu to about 10¹² pfu (e.g., about 10⁵ pfu to about 10¹² pfu, about 10⁶ pfu to about 10¹¹ pfu, or about 10⁶ pfu to about 10¹⁰ pfu). A therapeutically effective dose can be provided in repeated doses. Repeat dosing is appropriate in cases in which observations of clinical symptoms or tumor size or monitoring assays indicate either that a group of cancer cells or tumor has stopped shrinking or that the degree of viral activity is declining while the tumor is still present. Repeat doses can be administered by the same route as initially used or by another route. A therapeutically effective dose can be delivered in several discrete doses (e.g., days or weeks apart) and in one embodiment, one to about twelve doses are provided. Alternatively, a therapeutically effective dose of vesicular stomatitis viruses provided herein can be delivered by a sustained release formulation. In some cases, a vesicular stomatitis virus provided herein can be delivered in combination with pharmacological agents that facilitate viral replication and spread within cancer cells or agents that protect non-cancer cells from viral toxicity. Examples of such agents are described elsewhere (Alvarez-Breckenridge et al., Chem. Rev., 109(7):3125-40 (2009)).

Vesicular stomatitis viruses provided herein can be administered using a device for providing sustained release. A formulation for sustained release of vesicular stomatitis viruses can include, for example, a polymeric excipient (e.g., a swellable or non-swellable gel, or collagen). A therapeutically effective dose of vesicular stomatitis viruses can be provided within a polymeric excipient, wherein the excipient/virus composition is implanted at a site of cancer cells (e.g., in proximity to or within a tumor). The action of body fluids gradually dissolves the excipient and continuously releases the effective dose of virus over a period of time. Alternatively, a sustained release device can contain a series of alternating active and spacer layers. Each active layer of such a device typically contains a dose of virus embedded in excipient, while each spacer layer contains only excipient or low concentrations of virus (i.e., lower than the effective dose). As each successive layer of the device dissolves, pulsed doses of virus are delivered. The size/formulation of the spacer layers determines the time interval between doses and is optimized according to the therapeutic regimen being used.

Vesicular stomatitis viruses provided herein can be directly administered. For example, a virus can be injected directly into a tumor (e.g., a breast cancer tumor) that is palpable through the skin. Ultrasound guidance also can be used in such a method. Alternatively, direct administration of a virus can be achieved via a catheter line or other medical access device, and can be used in conjunction with an imaging system to localize a group of cancer cells. By this method, an implantable dosing device typically is placed in proximity to a group of cancer cells using a guidewire inserted into the medical access device. An effective dose of a vesicular stomatitis virus provided herein can be directly administered to a group of cancer cells that is visible in an exposed surgical field.

In some cases, vesicular stomatitis viruses provided herein can be delivered systemically. For example, systemic delivery can be achieved intravenously via injection or via an intravenous delivery device designed for administration of multiple doses of a medicament. Such devices include, but are not limited to, winged infusion needles, peripheral intravenous catheters, midline catheters, peripherally inserted central catheters, and surgically placed catheters or ports.

The course of therapy with a vesicular stomatitis virus provided herein can be monitored by evaluating changes in clinical symptoms or by direct monitoring of the number of cancer cells or size of a tumor. For a solid tumor, the effectiveness of virus treatment can be assessed by measuring the size or weight of the tumor before and after treatment. Tumor size can be measured either directly (e.g., using calipers), or by using imaging techniques (e.g., X-ray, magnetic resonance imaging, or computerized tomography) or from the assessment of non-imaging optical data (e.g., spectral data). For a group of cancer cells (e.g., leukemia cells), the effectiveness of viral treatment can be determined by measuring the absolute number of leukemia cells in the circulation of a patient before and after treatment. The effectiveness of viral treatment also can be assessed by monitoring the levels of a cancer specific antigen. Cancer specific antigens include, for example, carcinoembryonic antigen (CEA), prostate specific antigen (PSA), prostatic acid phosphatase (PAP), CA 125, alpha-fetoprotein (AFP), carbohydrate antigen 15-3, and carbohydrate antigen 19-4.

The invention will be further described in the following examples, which do not limit the scope of the invention described in the claims.

### EXAMPLES

### Example 1

### Producing a Vesicular Stomatitis Virus that Contains Maraba G Polypeptide

To produce replication-competent VSV-Maraba G (VSV-MG) viruses, a plasmid containing VSV (Indiana strain) full length genome was digested with restriction enzymes to remove VSV-G nucleic acid. Then, Maraba G nucleic acid (preceded by a VSV intergenic region like the rest of VSV genes) was cloned between VSV-M and VSV-L genes, and the viruses were rescued using techniques similar to those described elsewhere (Schnell et al., PNAS, 93:11359-11365 (1996), Obuchi et al., J. Virol., 77(16):8843-56 (2003)); Goel et al., Blood, 110(7):2342-50 (2007)); and Kelly et al., J. Virol., 84(3):1550-62 (2010)). Briefly, recombinant VSV/MarabaG virus was generated as follows. First, Maraba G was PCR amplified from Maraba virus (GenBank® Accession No. FN339541 or GI No. 2985638460) with primers VSV/Mar-F: 5′-T-3′ (SEQ ID NO:1) and VSV/Mar-1R: 5′-AATCTGTTGTGCAGGATTTGAGTTATT-3′ (SEQ ID NO:2), and VSV intergenic region was amplified from VSV (GenBank® Accession Nos. NC_{—}001560 or GI No. 9627229) using VSV/Mar-2F: 5′-GAGTCGATTGGGAAATAAATAACTCAA-3′(SEQ ID NO:3) and GFP68R: 5′-GCTGAACTTGTGGCCGTTTA-3′ (SEQ ID NO:4). An overlapping PCR using primers VSV/Mar-1R and GFP68R was performed. The overlapping PCR product was double digested with restriction enzymes MluI and AvrII and cloned into VSV plasmid vector (GenBank® Accession Nos. NC_{—}001560 (GI No. 9627229)), replacing VSV G with Maraba G. Positive clones were selected by PCR and confirmed with sequencing analysis for proper insertion of Maraba G in place of VSV G (FIG. 1).

The viruses were used to infect Vero cells, BHK cells, or myeloma cells. VSV-MG was replicated and was used to produce new infectious virions by itself

### Example 2

### Characterization of Vesicular Stomatitis Viruses that Contain Maraba G Polypeptides

For infectivity assay (FIG. 2), different cell types (4T1, BHK, LM1, TrampC1, Vero, and MPC11) were plated on 12-well plate (1×10⁵ cells/well) overnight. Then, cells were incubated with wild type VSV or VSV/MarabaG virus at MOI of 10.0 for 1 hour at 37° C. After 1 hour, cells were washed with PBS, and then the medium was replaced with DMEM plus 5% FBS. Fluorescent images were taken at 24 hour post-infection.

For virus growth curves (FIG. 3), Vero cells were incubated with VSV or VSV/MarabaG virus at an MOI of 1.0 for 1 hour at 37° C. Following this incubation, supernatant was removed, the monolayer was washed, and fresh growth medium was added. Supernatant was collected at predetermined time points (0, 4, 8, 12, 24, 48, 72 and 96 hours) and subjected to low-speed centrifugation, filtered through a 0.2 μm filter and titrated on Vero cells. For virus titration, Vero cells were grown on 96-well plates and infected with serially diluted virus stocks. Fifty percent tissue culture infectious dose (TCID₅₀/mL) values were determined.

For neutralization assays (FIGS. 4 and 5), wild type VSV or VSV/MarabaG or wild type Maraba virus (5 μL volume with 1×10⁷ TCID₅₀s of virus was incubated with 100 μL undiluted non-immune human serum (NHS) that contained 10% standard Guinea pig complement (Cedarlane) or VSV-immune serum at 37° C. for 1 hour, followed by determination of the virus titer (number of TCID₅₀s/mL) on Vero cells (1×10⁴ cells/well) (FIG. 5). As a control, virus was incubated with medium only and titrated on Vero cells. Neutralization assay results were read at 48 hours after infection. In addition, virus treated in the presence or absence of serum was plated on 12-well plates of Vero cells (1×10⁵ cells/well) and fluorescent (top) and phase-contrast (bottom) images were taken at 24 hours post-infection for each condition (FIG. 4).

In additional experiments, VSV/Maraba G was confirmed to resist neutralization, both by normal human serum (NHS) and VSV-immune sera from different origins (FIG. 6). Normal human serum inhibited VSV, but not VSV/MarG, infection in multiple cell lines (FIG. 7). Further, VSV, Maraba, and VSV/MarG viruses possess similar receptor requirements (FIG. 8).

In another experiment, mice immunized with adoptive transfer of 100 μL anti-VSV antibodies and having multiple myeloma were treated with PBS, wild-type VSV viruses (2×10⁸ TCID₅₀), or VSV/Maraba G (2×10⁸ TCID₅₀) viruses. The percent survival for each group of treated mice was determined Immunized multiple myeloma mice treated with VSV/MarG viruses exhibited superior survival as compared to immunized multiple myeloma mice treated with wild-type VSV viruses (FIG. 9).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A replication-incompetent vesicular stomatitis virus comprising a maraba G polypeptide and an RNA molecule, wherein said RNA molecule comprises a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, and a nucleic acid sequence that is a template for a positive sense transcript encoding a VSV L polypeptide, wherein said RNA molecule lacks a nucleic acid sequence that is a template for a positive sense transcript encoding a functional VSV G polypeptide.

2. The virus of claim 1, wherein said RNA molecule comprises a nucleic acid sequence that is a template for a positive sense transcript encoding a NIS polypeptide.

3. A nucleic acid molecule comprising a nucleic acid strand comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein said nucleic acid strand lacks a nucleic acid sequence that is a template for a positive sense transcript encoding a functional VSV G polypeptide.

4. The nucleic acid molecule of claim 3, wherein said nucleic acid strand comprises a nucleic acid sequence that is a template for a positive sense transcript encoding a NIS polypeptide.

5. The nucleic acid molecule of claim 4, wherein said NIS polypeptide is a human NIS polypeptide.

6. A method for treating cancer, wherein said method comprises administering a composition comprising replication-competent vesicular stomatitis viruses to a mammal comprising cancer cells, wherein said vesicular stomatitis viruses comprise an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein said RNA molecule lacks a nucleic acid sequence that is a template for a positive sense transcript encoding a functional VSV G polypeptide, wherein administration of said composition to said mammal is under conditions wherein said vesicular stomatitis viruses infect said cancer cells to form infected cancer cells, and wherein the number of cancer cells within said mammal is reduced following said administration.

7. The method of claim 6, wherein said mammal is a human.

8. The method of claim 6, wherein said RNA molecule comprises a nucleic acid sequence that is a template for a positive sense transcript encoding a NIS polypeptide.

9. The method of claim 8, wherein said NIS polypeptide is a human NIS polypeptide.

10. The method of claim 6, wherein said cancer is multiple myeloma.

11. A method for inducing tumor regression in a mammal, wherein said method comprises administering a composition comprising replication-competent vesicular stomatitis viruses to a mammal comprising a tumor, wherein said vesicular stomatitis viruses comprise an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a maraba G polypeptide, and a nucleic acid sequence that is a template for a positive sense transcript encoding a VSV L polypeptide, wherein said RNA molecule lacks a nucleic acid sequence that is a template for a positive sense transcript encoding a functional VSV G polypeptide, wherein administration of said composition to said mammal is under conditions wherein said vesicular stomatitis viruses infect tumor cells of said tumor to form infected tumor cells.

12. The method of claim 11, wherein said mammal is a human.

13. The method of claim 11, wherein said RNA molecule comprises a nucleic acid sequence that is a template for a positive sense transcript encoding a NIS polypeptide.

14. The method of claim 13, wherein said NIS polypeptide is a human NIS polypeptide.

15. The method of claim 11, wherein said tumor is multiple myeloma.
